(19) United States
(12) Patent Application Publication
(10) Pub. No.: US 2023/0165868 A1
(43) Pub. Date: Jun. 1, 2023

(54) PARAXANTHINE-BASED COMPOSITIONS FOR ENHANCING MUSCLE FUNCTION, NITRIC OXIDE SIGNALING, AND/OR MUSCLE GLYCOGEN LEVELS

(71) Applicant: Ingenious Ingredients, LP, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 18/070,435

(22) Filed: Nov. 28, 2022

Related U.S. Application Data
(60) Provisional application No. 63/283,515, filed on Nov. 28, 2021.
(60) Provisional application No. 63/284,994, filed on Dec. 1, 2021.
(60) Provisional application No. 63/291,145, filed on Dec. 17, 2021.

(51) Int. Cl. A61K 31/522; A61P 21/00; A61K 45/06

(52) U.S. Cl. CPC A61K 31/522; A61P 21/00; A61K 45/06

(57) ABSTRACT

Paraxanthine-based compositions for muscle enhancement. Paraxanthine-based compositions for enhancing nitrogen signaling. Paraxanthine-based compositions for enhancing muscle glycogen.

### CROSS-REFERENCE TO RELATED APPLICATION(S)

This application claims priority to U.S. Provisional Application No. 63/283,515 filed Nov. 28, 2021 and entitled “Paraxanthine-Based Compositions for Enhancing Muscle Function,” U.S. Provisional Application No. 63/284,994 filed Dec. 1, 2021 and entitled “Paraxanthine-Based Compositions for Enhancing Nitric Oxide Signaling,” and U.S. Provisional Application No. 63/291,145 filed Dec. 17, 2021 and entitled “Paraxanthine Based Compositions for Enhancing Muscle Glycogen Levels,” which are hereby incorporated by reference in their entirety under 35 U.S.C. § 119(e).

### TECHNICAL FIELD

The disclosed technology relates generally to compositions, methods, and system for utilizing paraxanthine alone and in combination for use in enhancing muscle function through administration of paraxanthine-containing enhancers of muscle function in a subject. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Paraxanthine is also known as 1,7-dimethylxanthine or 1,7-dimethyl-3H-purine-2,6-dione. Paraxanthine is structurally related to caffeine as well as a metabolite of caffeine which is also found through caffeine excretion in humans. In humans and other animals caffeine is first degraded to either paraxanthine, theobromine or theophylline, and then later, to a methylated xanthine.

Increasing muscle function is a key objective from professional athletes to fitness enthusiasts. Furthermore, preserving muscle function is critical to healthy aging. Muscle loss (atrophy) associated with aging, illness, or muscle denervation is associated with lower long term health. There is a need in the art for compositions and methods to increase muscle function and/or prevent muscle atrophy.

NO signaling has been implicated in a diverse array of physiological functions including neuronal signaling, immune response, inflammatory response, modulation of ion channels, phagocytic defense mechanism, penile erection, and cardiovascular homeostasis and its decompensation in atherogenesis. Increases in nitric oxide levels have been linked to increases in blood flow as well as aerobic exercise performance and cardiovascular health. Increased blood flow to working muscles can potentially improve sports performance, by increasing work efficiency, the modulation of force production and the reduction in phosphocreatine degradation. Accordingly, there is a need in the art for compositions and methods to enhance NO signaling.

Glycogen is a branched polymer of glucose that acts as a store of energy in times of nutritional sufficiency for utilization in times of need. In skeletal muscle, glycogen is found in a low concentration (1-2% of the muscle mass) and the skeletal muscle of an adult weighing 70 kg stores roughly 400 grams of glycogen. The amount of glycogen stored in the body—particularly within the muscles and liver—mostly depends on physical training, basal metabolic rate, and eating habit. Long-distance athletes, such as marathon runners, cross-country skiers, and cyclists, often experience glycogen depletion, where almost all of the athlete's glycogen stores are depleted after long periods of exertion without sufficient carbohydrate consumption. This depletion can lead the athlete to “hit the wall” which can significantly hinder the ability of the athlete to continue to perform. Furthermore, the restoration of muscle glycogen after depletion by exercise is a central component of the recovery process. There is a need in the art for compositions and methods to enhance and/or preserve muscle glycogen levels during endurance exercise.

### BRIEF SUMMARY

This disclosure relates to the use of a chemical composition comprising paraxanthine, either naturally or synthetically produced, and optionally other chemicals, including paraxanthine congeners or analogs, to provide a plurality of desirable effects. Further, disclosed herein is a nutritional supplement comprising about 2 mg to about 800 mg of paraxanthine.

The disclosure relates to compositions and methods of using such compositions for increasing muscle mass of a subject or for preventing muscle atrophy of a subject. The disclosure further relates to compositions and methods of using such compositions for enhancing the protein concentration or muscle mass of a mammal and a method for enhancing the protein concentration or muscle mass in a mammal. Furthermore, the disclosure relates to compositions and methods of using such compositions for increasing muscle mass of a subject or for preventing muscle atrophy of a subject.

The disclosure relates to compositions and methods of using such compositions for increasing NO signaling in a subject. The disclosure further relates to compositions and methods of using such compositions to enhance neuronal signaling, immune response, inflammatory response, modulation of ion channels, phagocytic defense mechanisms, penile erection, and/or cardiovascular homeostasis.

The disclosure relates to compositions and methods of using such compositions for increasing muscle glycogen levels and/or athletic endurance in a subject.

Disclosed herein is a method for increasing muscle function in a subject by providing the subject with a composition comprising an effective amount of paraxanthine. In certain embodiments, paraxanthine is present in the composition in amount from about 25 mg to about 600 mg. In further embodiments, paraxanthine is present in the composition in amount from about 50 mg to about 400 mg. In certain embodiments, the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, carnitine, creatinol, beta-alanine, ATP, protein, peptides, and beta-hydroxy beta-methylbutyrate.

According to certain implementations, the composition is substantially free of caffeine. In further implementations, the composition is substantially free of caffeine, taurine, tyrosine, and 1-methylxantine.

According to certain embodiments, the composition is administered in conjunction with a strength training regimen. In exemplary implementations, muscle function is increased by from about 10% to about 20%, relative to a subject receiving a control composition without paraxanthine.

Further disclosed herein is a method of increasing muscle strength and/or muscle size comprising administering to a subject in need thereof with a composition comprising an effective amount of paraxanthine. In certain implementations, muscle strength is increased from about 10-25% relative to a subject receiving a control composition without paraxanthine. In further implementations, muscle size is increased from about 10-20%, relative to a subject receiving a control composition without paraxanthine.

According to certain embodiments, nitric oxide (NO) signaling is increased from about 90-100% relative to a subject receiving a control composition without paraxanthine. In further embodiments, muscle glycogen levels are increased from about 10-30%, relative to a subject receiving a control composition without paraxanthine.

Further disclosed herein is a method of treating or preventing muscle atrophy in a subject in need thereof of comprising administering to the subject and effective amount of paraxanthine. In certain embodiments, the subject has been diagnosed with, or is at risk of developing, sarcopenia or cachexia.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows a chart for the results of the effect of supplementation on forelimb grip strength.

FIG. 2 shows distance traveled on treadmill in centimeters after 28 days of supplementation.

FIG. 3 shows muscle weights of gastrocnemius and soleus after 28 days of supplementation.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

As used herein, the terms “effective amount” and “amount effective” refer to an amount that is sufficient to achieve the desired result or effect (e.g., increasing muscle function).

As used herein, the terms “nutritional supplement” and “dietary supplement” refer to any product that is added to the diet. In some particularly preferred embodiments, nutritional supplements are taken by mouth and often contain one or more dietary ingredients, including but not limited to vitamins, minerals, herbs, amino acids, enzymes, and cultures of organisms.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a muscle atrophy disorder” means having been subjected to a physical examination by a person of skill, for example, a physician, and found to have a condition that can be diagnosed or treated by a compound or composition that can increase muscle mass. As a further example, “diagnosed with a need for increasing muscle mass” refers to having been subjected to a physical examination by a person of skill, for example, a physician, and found to have a condition characterized by muscle atrophy or other disease wherein increasing muscle mass would be beneficial to the subject. Such a diagnosis can be in reference to a disorder, such as muscle atrophy, and the like, as discussed herein. As a further example, “diagnosed with a need for increasing nitric oxide signaling” refers to having been subjected to a physical examination by a person of skill, for example, a physician, and found to have a condition characterized by low levels of neuronal signaling, immune response, inflammatory response, modulation of ion channels, phagocytic defense mechanism, penile erection, and/or cardiovascular homeostasis.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to muscle atrophy, a disorder related to low NO signaling, or a disorder related to muscle atrophy) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

In certain aspects, disclosed herein are methods to promote muscle growth through the administration of an effective amount of one or more compositions disclosed herein. In certain further aspects, administration of effective amounts of the disclosed compositions results in greater level of muscle protein synthesis (MPS) in the subject. In still further aspects, administration of effective amounts of the disclosed compositions results in improved muscle accretion in the subject.

According to certain embodiments, compositions disclosed herein may be administered in conjunction with a strength training regime. As will be appreciated by a person having skill in the art, administration of effective amounts of the disclosed compositions results in improved strength and improved athletic performance/ergogenesis in the subject.

In exemplary implementations, muscle function is increased by from about 5% to about 30%, relative to a subject receiving a control composition without paraxanthine. In further implementations, muscle function is increased by from about 10% to about 20%, relative to a subject receiving a control composition without paraxanthine. In yet further implementations, muscle function is increased by about 15% relative to a subject receiving a control composition without paraxanthine

In certain implementations, muscle strength is increased from about 5% to about 35% relative to a subject receiving a control composition without paraxanthine. In further embodiments, muscle strength is increased from about 10% to about 25% relative to a subject receiving a control composition without paraxanthine. In yet further embodiments, muscle strength is increased by about 15% relative to a subject receiving a control composition without paraxanthine.

In further implementations, muscle size (e.g. muscle mass) is increased from about 5-30%, relative to a subject receiving a control composition without paraxanthine. In further embodiments, muscle size is increased from about 10% to about 25% relative to a subject receiving a control composition without paraxanthine. In yet further embodiments, muscle size is increased by about 15% relative to a subject receiving a control composition without paraxanthine

According to certain embodiments, nitric oxide (NO) signaling is increased from about 80-110% relative to a subject receiving a control composition without paraxanthine. In further embodiments, NO signaling is increased from about 95-105% relative to a subject receiving a control composition without paraxanthine. In still further embodiments, NO signaling is increased about 100% relative to a subject receiving a control composition without paraxanthine.

In further embodiments, muscle glycogen levels are increased from about 5-30%, relative to a subject receiving a control composition without paraxanthine. In further embodiments, muscle glycogen is increased from about 10% to about 25% relative to a subject receiving a control composition without paraxanthine. In yet further embodiments, muscle glycogen is increased by about 15% relative to a subject receiving a control composition without paraxanthine

In certain aspects, disclosed herein are methods to promote NO signaling through the administration of an effective amount of one or more compositions disclosed herein.

In one aspect, the disclosed compounds reduce hypertension. In a further aspect, the disclosed compounds increase vasodilation. In a still further aspect, the disclosed compounds increase cardiac homeostasis. In a still further aspect, the subject is a mammal. In a yet further aspect, the mammal is a human.

In one aspect, the disclosed compounds inhibit muscle atrophy. In a further aspect, the disclosed compounds increase muscle mass. In a still further aspect, the disclosed compounds induce muscle hypertrophy. In a yet further aspect, the disclosed compounds inhibit of muscle atrophy and increase muscle mass. In an even further aspect, the disclosed compounds inhibit of muscle atrophy and induce muscle hypertrophy. In a further aspect, the inhibition of muscle atrophy is in a subject. In an even further aspect, the increase in muscle mass is in a subject. In a still further aspect, the subject is a mammal. In a yet further aspect, the mammal is a human.

In certain aspects, administration of the disclosed compositions is effective at preventing or treating age-related muscle atrophy or sarcopenia. In further aspects, administration of the disclosed compositions is effective at preventing or treating muscle atrophy associated with muscle immobilization, such as that which frequently occurs with casting of fractured bones. In further aspects, administration of the disclosed compositions is effective at preventing or treating muscle atrophy associated with disease, such as cancer, also known as cachexia. In further aspects, increases blood flow to the muscle. In further aspects, increases blood flow to the muscle in an elderly subject.

According to certain aspects the composition is administered to a subject that has sarcopenia. In various aspects, the composition is administered in a therapeutically effective amount. In further aspects, the composition is administered at prophylactically effective amount, (e.g. to a subject at risk for developing sarcopenia, cachexia, or immobilization induced atrophy).

Disclosed herein is a method for prevention or treating muscle atrophy in a subject, the method comprises administering to the subject an effective amount of a composition comprising from about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain aspects, disclosed herein are methods to enhancing muscle glycogen levels through the administration of an effective amount of one or more compositions disclosed herein (e.g. a composition comprising from about 2 mg to about 800 mg of paraxanthine). In certain embodiments, administration of the disclosed compositions enhances baseline levels of glycogen stored in the muscle (e.g. glycogen stored in the muscle when at rest). In further embodiments, administration of the disclosed composition reduces the rate at which glycogen is depleted during exercise. In certain exemplary implementations of these embodiments, the composition is administered prior to the beginning of exercise. In further implementations, the composition is administered during exercise. In still further implementations, the composition is administered both before and during exercise.

In further embodiments, administration of the composition increases the rate at which exercise-depleted glycogen is restored in the muscle following exercise. In such embodiments, the rate of recovery of the subject may be significantly increased. In exemplary implementations of these embodiments, the disclosed composition is administered immediately following exercise. In further embodiments, the composition is administered both during and immediately following exercise.

In some embodiments, the subjects to be treated includes subjects in need of treatment of muscle glycogen depletion due to exhaustive exercise. Examples of such subjects include athletes, such as endurance and team sports athletes as well as athletes participating in weight class regulated sports. Other examples include professional cyclists and professional football player and ice hockey players.

In some embodiments, the subjects to be treated includes subjects in need of treatment of loss of muscle mass. Examples of such human beings include elderly people. Other examples include elderly people, who have been bedbound for a period due to illness or surgery. Other examples include elderly people, who have not been physical active for a period. In yet other embodiments, the group of human beings to be treated includes subjects of all ages during their period of rehabilitation. In other embodiments the group to be treated includes elderly people during their period of rehabilitation.

Further disclosed herein is a method for enhancing muscle glycogen levels in a subject, the method comprises administering to the subject an effective amount of a composition comprising from about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain aspects, the composition further comprises one or more additional active ingredient to further enhance muscle strength, size, and/or muscle function. In certain embodiments, the one or more additional active ingredient is an amino acid. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, and tyrosine. According to the certain embodiments, the conditionally essential amino acid is tyrosine. In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, creatinol, beta-alanine, taurine, beta-hydroxy beta-methylbutyrate L-Arginine, omega-3 fatty acids, Vitamin D, Non-Steroidal Anti-Inflammatory Drugs (NSAID), whey protein, and other protein extracts from animal, plant or fermentation sources. In further embodiments,

In still further embodiments, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

According to certain embodiments, the composition administered in any of the foregoing methods is formulated such that a dose contains paraxanthine each ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) and taurine ranging from 400 to about 3000 mg (e.g., about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg and the like, or any range or value therein).

According to certain embodiments, the composition administered in any of the foregoing methods is substantially free of caffeine. In further embodiments, the composition is substantially free of tyrosine. In yet further embodiments, the composition is substantially free of taurine. In still further embodiments, the composition is substantially free of 1-methylxantine. In further embodiments, the composition is substantially free of 7-methylxantine. In still further embodiments, the composition is substantially free of each of caffeine, taurine, tyrosine, 1-methylxantine, and 7-methylxantine. In yet further embodiments, any of the method disclosed herein, the subject abstains from consuming one or more of caffeine, taurine, tyrosine, 1-methylxantine, and 7-methylxantine during performance of the method.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:

1. A method for increasing muscle function in a subject, comprising: providing the subject with a composition comprising about 2 mg to about 800 mg of paraxanthine.
2. The method of clause 1, wherein paraxanthine is present in the composition in amount from about 20 mg to about 600 mg.
3. The method of clause 2, wherein paraxanthine is present in the composition in amount from about 50 mg to about 400 mg.
4. The method of any of clauses 1-3, wherein the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, and beta-hydroxy beta-methylbutyrate.
5. The method of any preceding clause wherein the paraxanthine is derived from a natural source.
6. The method of any preceding clause wherein the paraxanthine is synthetic.
7. A nutritional supplement for improving muscle strength, muscle size, and/or muscle function comprising from about 2 mg to about 800 mg paraxanthine, either natural or synthetic.
8. The nutritional supplement of clause 7 wherein the paraxanthine is present in amount from about 20 mg to about 600 mg.
9. The nutritional supplement of clause 7 wherein the paraxanthine is present in amount from about 50 mg to about 400 mg.
10. The nutritional supplement of any of clauses 7-9, wherein the nutritional supplement is a dietary supplement.
11. The nutritional supplement of clause 10, wherein the nutritional supplement is powder or a capsule.
12. The nutritional supplement any of clauses 7-9, wherein the nutritional supplement is a functional food.
13. The nutritional supplement of clause 12, wherein the functional food is a beverage, nutrition bar, yoghurt, or cereal.
14. The nutritional supplement any of clauses 7-9, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
15. The nutritional supplement of any of clauses 7-14, wherein the paraxanthine is derived from a natural source.
16. The nutritional supplement of any of clauses 7-14, wherein the paraxanthine is synthetic.
17. A method of increasing muscle strength, muscle size, and/or muscle function comprising administering to a subject in need thereof with a composition comprising about 2 mg to about 800 mg of paraxanthine.
18. The method of clause 17, wherein paraxanthine is present in the composition in amount from about 20 mg to about 600 mg.
19. The method of clause 2, wherein paraxanthine is present in the composition in amount from about 50 mg to about 400 mg.
20. The method of any of clauses 17-19, wherein the subject has been diagnosed with sarcopenia.
21. The method of any of clauses 17-19, wherein the subject is at risk of developing sarcopenia.
22. The method of any of clauses 17-19, wherein the subject has been diagnosed with cachexia.
23. The method of any of clauses 17-19, wherein the subject is at risk of developing cachexia.
29. The method of any preceding clause wherein the paraxanthine is synthetic.
30. A nutritional supplement for improving muscle strength, muscle size, and/or muscle function comprising from about 2 mg to about 800 mg paraxanthine, either natural or synthetic.
31. The nutritional supplement of clause 30 wherein the paraxanthine is present in amount from about 20 mg to about 600 mg.
32. The nutritional supplement of clause 30 wherein the paraxanthine is present in amount from about 50 mg to about 400 mg.
33. The nutritional supplement of any of clauses 30-32, wherein the nutritional supplement is a dietary supplement.
34. The nutritional supplement of clause 33, wherein the dietary supplement is powder or a capsule.
35. The nutritional supplement any of clauses 30-32, wherein the nutritional supplement is a functional food.
36. The nutritional supplement of clause 35, wherein the functional food is a beverage, nutrition bar, yoghurt, or cereal.
37. The nutritional supplement any of clauses 30-32, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
38. The nutritional supplement of any of clauses 30-37, wherein the paraxanthine is derived from a natural source.
39. The nutritional supplement of any of clauses 30-37, wherein the paraxanthine is synthetic.
40. A method of increasing muscle strength, muscle size, and/or muscle function comprising administering to a subject in need thereof with a composition comprising about 2 mg to about 800 mg of paraxanthine.
41. The method of clause 40, wherein paraxanthine is present in the composition in amount from about 20 mg to about 600 mg.
42. The method of clause 25, wherein paraxanthine is present in the composition in amount from about 50 mg to about 400 mg.
43. The method of any of clauses 40-42, wherein the subject has been diagnosed with sarcopenia.
44. The method of any of clauses 40-42, wherein the subject is at risk of developing sarcopenia.
45. The method of any of clauses 40-42, wherein the subject has been diagnosed with cachexia.
46. The method of any of clauses 40-42, wherein the subject is at risk of developing cachexia.
47. A method for enhancing muscle glycogen levels in a subject, comprising:
providing the subject with a composition comprising about 2 mg to about 800 mg of paraxanthine.
48. The method of clause 47, wherein paraxanthine is present in the composition in amount from about 20 mg to about 600 mg.
49. The method of clause 48, wherein paraxanthine is present in the composition in amount from about 50 mg to about 400 mg.
50. The method of any of clauses 47-49, wherein the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, and beta-hydroxy beta-methylbutyrate.
51. The method of any preceding clause wherein the paraxanthine is derived from a natural source.
52. The method of any preceding clause wherein the paraxanthine is synthetic.
53. A nutritional supplement for enhancing muscle glycogen levels comprising from about 2 mg to about 800 mg paraxanthine, either natural or synthetic.
54. The nutritional supplement of clause 53 wherein the paraxanthine is present in amount from about 20 mg to about 600 mg.
55. The nutritional supplement of clause 53 wherein the paraxanthine is present in amount from about 50 mg to about 400 mg.
56. The nutritional supplement of any of clauses 53-55, wherein the nutritional supplement is a dietary supplement.
57. The nutritional supplement of clause 56, wherein the nutritional supplement is powder or a capsule.
58. The nutritional supplement any of clauses 53-55, wherein the nutritional supplement is a functional food.
59. The nutritional supplement of clause 58, wherein the functional food is a beverage, nutrition bar, yoghurt, or cereal.
60. The nutritional supplement any of clauses 53-55, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
61. The nutritional supplement of any of clauses 53-60, wherein the paraxanthine is derived from a natural source.
62. The nutritional supplement of any of clauses 53-60, wherein the paraxanthine is synthetic.
63. A method for enhancing restoration of muscle glycogen levels after depletion by exercise in subject in need thereof comprising administering to the subject a composition comprising about 2 mg to about 800 mg of paraxanthine.
64. The method of clause 63, wherein paraxanthine is present in the composition in amount from about 20 mg to about 600 mg.
65. The method of clause 48, wherein paraxanthine is present in the composition in amount from about 50 mg to about 400 mg.
66. The method of any preceding clause wherein the paraxanthine is synthetic.
67. A nutritional supplement for improving muscle strength, muscle size, and/or muscle glycogen levels comprising from about 2 mg to about 800 mg paraxanthine, either natural or synthetic.
68. The nutritional supplement of clause 67 wherein the paraxanthine is present in amount from about 20 mg to about 600 mg.
69. The nutritional supplement of clause 67 wherein the paraxanthine is present in amount from about 50 mg to about 400 mg.
70. The nutritional supplement of any of clauses 67-69, wherein the nutritional supplement is a dietary supplement.
71. The nutritional supplement of clause 70, wherein the dietary supplement is powder or a capsule.
72. The nutritional supplement any of clauses 67-69, wherein the nutritional supplement is a functional food.
73. The nutritional supplement of clause 72, wherein the functional food is a beverage, nutrition bar, yoghurt, or cereal.
74. The nutritional supplement any of clauses 67-69, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
75. The nutritional supplement of any of clauses 67-74, wherein the paraxanthine is derived from a natural source.
76. The nutritional supplement of any of clauses 67-74, wherein the paraxanthine is synthetic.
77. A method of reducing the rate of muscle glycogen depletion in an exercising subject comprising administering to the subject a composition comprising about 2 mg to about 800 mg of paraxanthine.
78. The method of clause 77, wherein paraxanthine is present in the composition in amount from about 20 mg to about 600 mg.
79. The method of clause 77, wherein paraxanthine is present in the composition in amount from about 50 mg to about 400 mg.
80. The method of any of clauses 77-79, wherein the composition is administered prior to the beginning of exercise.
81. The method of any of clauses 77-79, wherein the composition is administered during exercise.
82. The method of clause 77, wherein the subject experiences increased endurance relative to performing the exercise without administration of the composition.
83. A method for enhancing nitric oxide signaling in a subject, comprising: providing the subject with a composition comprising about 2 mg to about 800 mg of paraxanthine.
84. The method of clause 83, wherein paraxanthine is present in the composition in amount from about 20 mg to about 600 mg.
85. The method of clause 84, wherein paraxanthine is present in the composition in amount from about 50 mg to about 400 mg.
86. The method of any of clauses 83-85, wherein the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, and beta-hydroxy beta-methylbutyrate.
87. The method of any preceding clause wherein the paraxanthine is derived from a natural source.
88. The method of any preceding clause wherein the paraxanthine is synthetic.
89. A nutritional supplement for enhancing NO signaling in a subject comprising from about 2 mg to about 800 mg paraxanthine, either natural or synthetic.
90. The nutritional supplement of clause 89 wherein the paraxanthine is present in amount from about 20 mg to about 600 mg.
91. The nutritional supplement of clause 89 wherein the paraxanthine is present in amount from about 50 mg to about 400 mg.
92. The nutritional supplement of any of clauses 89-91, wherein the nutritional supplement is a dietary supplement.
93. The nutritional supplement of clause 92, wherein the nutritional supplement is powder or a capsule.
94. The nutritional supplement any of clauses 89-91, wherein the nutritional supplement is a functional food.
95. The nutritional supplement of clause 94, wherein the functional food is a beverage, nutrition bar, yoghurt, or cereal.
96. The nutritional supplement any of clauses 89-91, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
97. The nutritional supplement of any of clauses 89-96, wherein the paraxanthine is derived from a natural source.
98. The nutritional supplement of any of clauses 89-96, wherein the paraxanthine is synthetic.
99. A method of increasing muscle function, cognition, cardiovascular health and/or sexual health in a subject in need thereof, comprising administering to the subject a composition comprising about 2 mg to about 800 mg of paraxanthine.
100. The method of clause 99, wherein paraxanthine is present in the composition in amount from about 20 mg to about 600 mg.
101. The method of clause 84, wherein paraxanthine is present in the composition in amount from about 50 mg to about 400 mg.
102. The method of any of clauses 99-101, wherein the subject has been diagnosed with sarcopenia.
103. The method of any of clauses 99-101, wherein the subject is at risk of developing sarcopenia.
104. The method of any of clauses 99-101, wherein the subject has been diagnosed hypertension.
105. The method of any of clauses 99-101, wherein the subject is at risk of developing hypertension.
106. The method of any preceding clause wherein the paraxanthine is synthetic.
107. A nutritional supplement for improving sexual health comprising from about 2 mg to about 800 mg paraxanthine, either natural or synthetic.
108. The nutritional supplement of clause 107 wherein the paraxanthine is present in amount from about 20 mg to about 600 mg.
109. The nutritional supplement of clause 107 wherein the paraxanthine is present in amount from about 50 mg to about 400 mg.
110. The nutritional supplement of any of clauses 107-109, wherein the nutritional supplement is a dietary supplement.
111. The nutritional supplement of clause 110, wherein the dietary supplement is powder or a capsule.
112. The nutritional supplement any of clauses 107-109, wherein the nutritional supplement is a functional food.
113. The nutritional supplement of clause 112, wherein the functional food is a beverage, nutrition bar, yoghurt, or cereal.
114. The nutritional supplement any of clauses 107-109, further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, tyrosine, carnitine, creatinol, beta-alanine, taurine, beta-hydroxy beta-methylbutyrate, creatine, L-arginine, Omega-3 fatty acids, Vitamin D, Non-Steroidal Anti-Inflammatory Drugs (NSAID), whey protein, and other protein extracts from animal, plant or fermentation sources.
115. The nutritional supplement of any of clauses 107-114, wherein the paraxanthine is derived from a natural source.
116. The nutritional supplement of any of clauses 107-114, wherein the paraxanthine is synthetic.
117. A method for enhancing vasodilation in a subject in need thereof comprising administering to the subject administering to the subject a composition comprising about 2 mg to about 800 mg of paraxanthine.
118. The method of clause 117, wherein paraxanthine is present in the composition in amount from about 20 mg to about 600 mg.
119. The method of clause 117, wherein the subject is suffering from hypertension.
120. The method or nutritional supplement of any preceding clause, wherein the composition or nutritional supplement is substantially free of caffeine.
121. The method or nutritional supplement of any preceding clause, wherein the composition or nutritional supplement is substantially free any or all of caffeine, taurine, tyrosine, 1-methylxantine, and/or 7-methylxantine.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Animals and Experiment Design

Forty 8-week-old male Swiss Albino mice were housed in an animal room at a constant temperature (22±3° C.) and humidity (30%-70%) under a 12:12 h light-dark cycle with standard laboratory diet (Purina 5L79, Rat and Mouse 18% protein; PMI Nutrition International, Brentwood, Mo., USA). Distilled water was provided ad libitum. All animal experiments were reviewed and approved by the Institutional Animal Ethical Committee (IAEC) of Radiant Research Services Pvt. Ltd (Bangalore, India). All research was conducted in accordance with the guidelines of the committee for the purpose of control and supervision of experiments on animals (CPCSEA Registration Number-1803/PO/RcBi/S/2015/CPCSEA).

After one week of acclimation, the animals were randomly divided by body weight into five groups (n=8 per group in each test) for oral treatment once a day, at approximately same time each day (±1 hour), for 28 consecutive days: (1) vehicle control; (2) paraxanthine (3) L-theanine; (4) alpha-GPC; and (5) taurine. The dose administered to the mice was calculated using US Food and Drug Administration for human equivalence doses (HED), assuming a human weight of 60 kg. The following HED were used in this study: 100 mg paraxanthine, (ENFINITY™, Ingenious Ingredients, L.P Lewisville, Tex., USA; mouse dose: 20.5 mg/kg bw/day); 200 mg alpha-GPC mg (YangLing Daily Health Bio-Engineering Technology Co., Ltd; mouse dose: 41.09 mg/kg bw), 50 mg L-theanine (Hangzhou Qinyuan Natural Plant High-tech Co., Ltd; mouse dose: 10.28 mg/kg bw), and 500 mg taurine (Jiangyin Huachang Food Additive Co., Ltd; mouse dose: 102.75 mg/kg bw). 0.5% Carboxy Methyl Cellulose sodium was used as vehicle and the test item formulations were prepared daily. Dosing was conducted via oral gavage using disposable polypropylene syringes with sterilized stainless steel gavage tubes. The food intake and water consumption were monitored daily, and BW was recorded weekly. The last dose on day 28 was given 1 hour prior to testing.

### Sample Collection

All animals were euthanized by 95% CO2 after 28 days of following their assigned treatment. Blood was collected immediately after euthanization by the retro-orbital route and was immediately centrifuged at 1500×g for 10 min at 4° C. before having all serum transferred into cryogenic tubes and stored at −80° C. The liver, heart, gastrocnemius and soleus were excised and weighed.

### Forelimb Grip Strength Test

The forelimb grip strength was measured on day 0 and day 28 by using a stainless-steel grill to assess muscle strength (Orchid Scientific & Innovative India Pvt Ltd, India). Grip strength was measured one hour after treatment. Briefly, each mouse was placed on a grill chamber and the steel bar is inverted; grip strength is measured by recording the time each animal holds the steel bar. The maximum time for each trial was six minutes. Each animal performed three independent trials separated by six minutes intervals and the mean of the three trials was calculated.

### Exercise Training

During the treatment period, exercise training was completed using a motorized treadmill (Exer 3/6, Columbus Instruments international, OH, USA) at a moderate intensity of 20 cm/sec as maximal running speed, an incline of ten degrees and a shock intensity of 0.2 mA, for ten minutes. The speed of the treadmill was manually adjusted by increasing the belt speed by 5 cm/sec every two minutes throughout the total duration of ten minutes. All animals were adapted to this procedure daily 60 minutes after dosing for five days in a week during the treatment period.

### Treadmill Endurance Test

On 28th day of each respective treatment, all animals were subjected to a muscle endurance test. Muscle endurance was accomplished on a motorized treadmill at a low to moderate intensity of 5-50 cm/sec as maximal running speed, an incline of ten degrees and a shock intensity of 0.2 mA, with the belt speed being increased by 5 cm/sec every two minutes until it reaches 50 cm/sec. Animals were subjected to the treadmill test until exhaustion. Distance traveled (cm) was measured as a marker of exercise performance.

### Clinical Biochemical Profiles

Collected serum was analyzed for clinical biochemical variables including aspartate aminotransaminase (AST), alanine aminotransaminase (ALT), alkaline phosphatase (ALP), uric acid (UA), creatinine, total cholesterol (TC), triglycerides (TG), high-density lipoproteins (HDL), and low-density lipoproteins (LDL) were measured using an auto analyzer (EM360, ERBA Diagnostics Mannheim GmbH, Mannheim, Germany). Nitric oxide was analyzed using standard ELISA assay kits (Lot No: E-BC-K035-M, Elabscience, Houston, Tex., USA).

### Statistical Analysis

Data are presented as mean±standard deviation (SD). Statistical differences among groups were analyzed using one-way ANOVA followed by the Dunnett's Test identify the pairs with significant differences using GraphPad PRISM Software, Version 5.01 (GraphPad Software, Inc. California, USA). The level of statistical significance was set at p<0.05.

### Results

### Effect on Body Weight and Feed Consumption

The initial and final body weights and food consumption did not significantly (p>0.05) differ between groups. The body weight is summarized in Table 1, and feed consumption in Table 2.

TABLE 1
Effect of test substance on body weight (in grams).

| Treatment | Basal | Week 1 | Week 2 | Week 3 | Week 4 |
|---|---|---|---|---|---|
| Control | 22.05 ± 0.65 | 23.90 ± 0.61 | 26.91 ± 0.55 | 29.61 ± 0.57 | 32.89 ± 0.33 |
| Paraxanthine | 22.01 ± 0.40 | 23.86 ± 0.55 | 26.95 ± 0.41 | 29.68 ± 0.45 | 32.80 ± 0.48 |
| L-Theanine | 22.41 ± 0.49 | 24.20 ± 0.56 | 26.76 ± 0.71 | 29.00 ± 0.60 | 31.60 ± 0.64 |
| Alpha-GPC | 22.50 ± 0.44 | 24.10 ± 0.40 | 27.05 ± 0.50 | 29.14 ± 0.49 | 31.68 ± 0.44 |
| Taurine | 22.50 ± 0.49 | 24.08 ± 0.66 | 26.99 ± 0.82 | 29.06 ± 0.69 | 32.34 ± 3.06 |

Data are mean ± SD for n = 8 mice in each group.

TABLE 2
Effect of test substance on feed consumption (in grams).

| Treatment | Week 1 | Week 2 | Week 3 | Week 4 |
|---|---|---|---|---|
| Control | 41.63 ± 0.73 | 43.65 ± 0.56 | 45.68 ± 0.68 | 47.83 ± 0.59 |
| Paraxanthine | 41.73 ± 046 | 43.89 ± 0.68 | 46.00 ± 0.59 | 48.34 ± 0.68 |
| L-Theanine | 41.26 ± 0.86 | 43.50 ± 0.66 | 45.30 ± 0.48 | 47.60 ± 0.51 |
| Alpha-GPC | 40.91 ± 0.55 | 43.34 ± 0.40 | 45.49 ± 0.40 | 47.54 ± 0.42 |
| Taurine | 40.95 ± 0.60 | 43.21 ± 1.25 | 45.21 ± 1.15 | 47.36 ± 1.08 |

Data are mean ± SD for n = 8 mice in each group.

### Effect of Supplementation on Forelimb Grip Strength

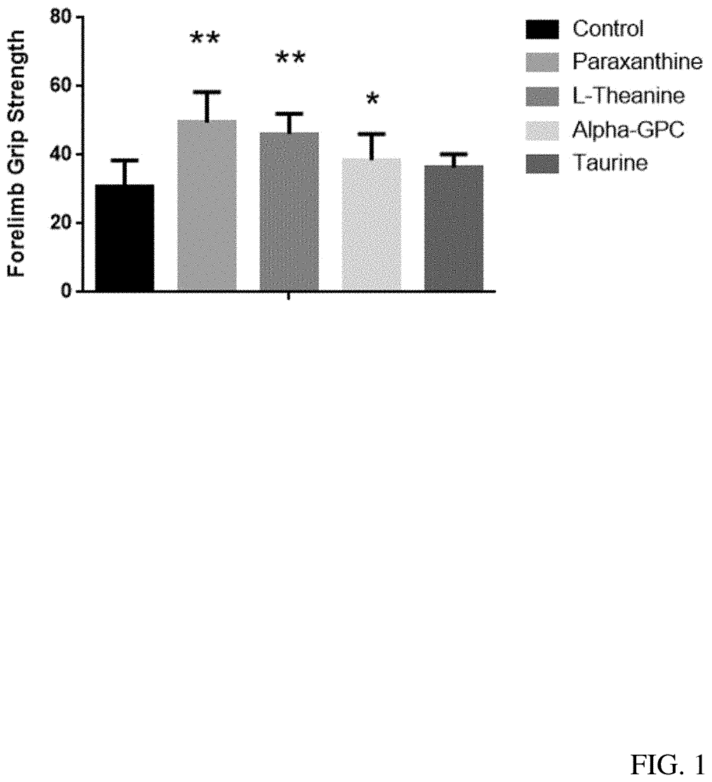

Baseline forelimb grip strength was not different between groups (control 88.94±1.75, paraxanthine 90.60±0.92, L-Theanine 87.90±2.63, alpha-GPC 91.00±0.40, taurine 90.94±0.85). When compared to control, paraxanthine supplementation significantly increased forelimb grip strength by 17% (p<0.001). Similarly, L-Theanine increased strength by 12% (p<0.001), and alpha-GPC by 8% (p<0.05), while improvements in the taurine group failed to reach statistical significance (p>0.05), see FIG. 1. Paraxanthine showed a significantly greater increase in grip strength, relative to other treatment groups. P<0.001 strength mass PX vs. Taurine, vs. P<0.05 alpha-GPC.

### Effect of Supplementation on Treadmill Performance

Baseline treadmill performance was not different between groups (control 267.1±29.3 cm, paraxanthine 255.3±30.7 cm, L-Theanine 265.4±32.9 cm, alpha-GPC 248.6±42.2 cm, taurine 256.9±29.3 cm). Paraxanthine supplementation significantly increased distance traveled during the treadmill exercise by 39% when compared to control (2,100±133 vs.

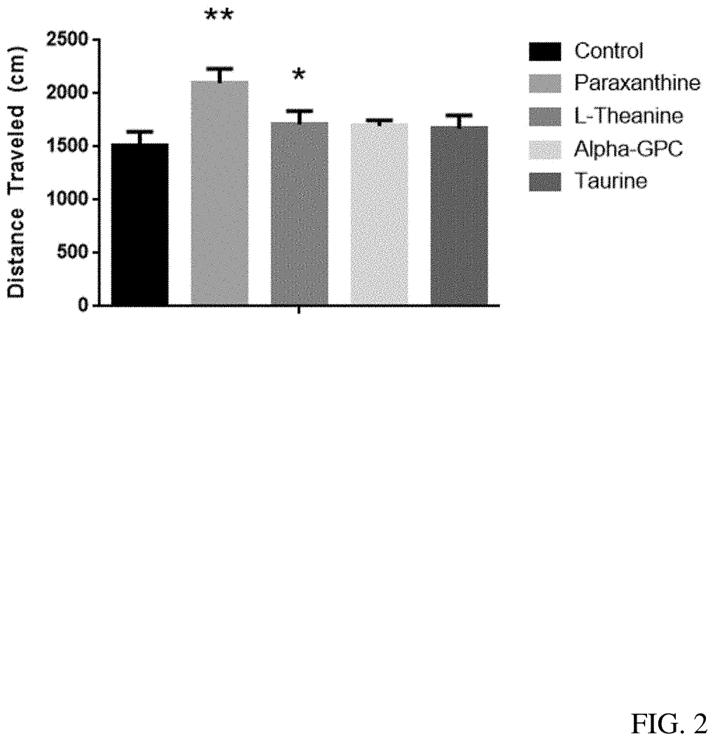

1,514±126, p<0.001). L-Theanine increased distanced traveled by 13% when compared to control (1,710±127, p<0.05). Taurine (1,671±125) and alpha-GPC (1,695±53.0) did not increase treadmill performance (p>0.05), FIG. 2 shows Distance traveled on treadmill in centimeters after 28 days of supplementation. **p<0.001 when compared to control. *p<0.05 when compared to control. Data is represented by mean±SD (n=8 in each group). P<0.001 treadmill performance Px vs. L-theanine, vs. alpha-GPC, vs. Taurine.

### Effect of Supplementation on Biochemical Markers of Health

Neither treatment had any effect (p>0.05) on markers of liver health (AST, ALT and ALP) and renal function (urea, creatinine), see Table 3.

TABLE 3
Biochemical analysis at the end of treatment.

| | AST | ALT | ALP | Urea | Creatinine |
|---|---|---|---|---|---|
| Treatment | (U/L) | (U/L) | (U/L) | (mg/dL) | (mg/dL) |
| Control | 41.13 ± 2.53 | 25.13 ± 1.13 | 180.8 ± 3.54 | 30.13 ± 1.36 | 0.96 ± 0.13 |
| Paraxanthine | 39.50 ± 1.60 | 23.88 ± 0.99 | 179.4 ± 2.56 | 29.13 ± 0.99 | 0.86 ± 0.11 |
| L-Theanine | 40.25 ± 2.44 | 24.25 ± 1.28 | 177.1 ± 3.68 | 28.75 ± 1.28 | 0.89 ± 0.11 |
| Alpha-GPC | 38.88 ± 2.10 | 23.63 ± 1.51 | 179.3 ± 3.99 | 29.38 ± 1.41 | 0.90 ± 0.16 |
| Taurine | 40.13 ± 2.36 | 25.00 ± 1.07 | 180.4 ± 5.42 | 29.38 ± 1.60 | 0.93 ± 0.14 |

Data are the mean ± SD for n = 8 mice in each group.
AST: aspartate aminotransferase; ALT: alanine aminotransferase; ALP: alkaline phosphatase.

### Effect of Supplementation of Muscle and Organ Weight

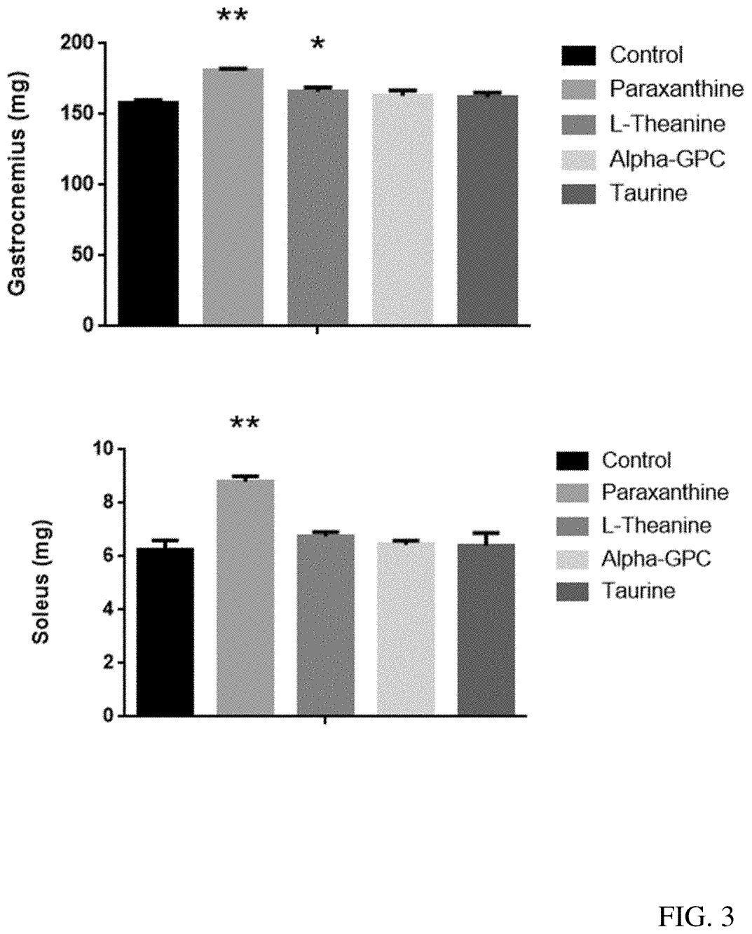

When compared to control, paraxanthine supplementation significantly increased gastrocnemius by 14% and soleus muscle mass by 41%, *p<0.001. L-Theanine improved gastrocnemius muscle mass by 5% (p<0.05), but not soleus muscle mass (p>0.05) when compared to control. Alpha-GPC and taurine supplementation had no effect on muscle mass. FIG. 3 shows Muscle weights of gastrocnemius and soleus after 28 days of supplementation. *p<0.05, *p<0.001 when compared to control. Date represented by Mean±SD (n=8 in each group). P<0.001 Soleus muscle mass PX vs. L-theanine, vs. alpha-GPC, vs. Taurine. P<0.001 Soleus gastrocnemius mass PX vs. L-theanine, vs. alpha-GPC, vs. Taurine.

Gross pathology evaluation of the heart and liver showed no changes with either of the treatments, see Table 4.

TABLE 4
Liver and heart mass in each group.

| Treatment | Liver (mg) | Heart (mg) |
|---|---|---|
| Control | 1,874 ± 25 | 189 ± 1.9 |
| Paraxanthine | 1,870 ± 31 | 190 ± 1.4 |
| L-Theanine | 1,866 ± 43 | 189 ± 2.3 |
| Alpha-GPC | 1,869 ± 32 | 189 ± 1.0 |
| Taurine | 1,834 ± 52 | 190 ± 1.2 |

Data Represented by Mean ± SD (n = 8 in each group).
No changes notes in all groups.

### Effect of Supplementation on NO Signaling

Paraxanthine significantly increased nitric oxide levels by 101% over control, while L-Theanine and alpha-GPC increased nitric oxide by 57%, and 42% over control, respectively (see Table 5). *p<0.05 vs. control, **p<0.001 vs. control.

TABLE 5
Effect on nitric oxide levels.

| Treatment | Nitric Oxide (ng/mL) |
|---|---|
| Control | 6.50 ± 0.40 |
| Paraxanthine | 13.06 ± 0.58** |
| L-Theanine | 10.23 ± 1.26** |
| Alpha-GPC | 9.21 ± 2.33** |
| Taurine | 7.56 ± 0.29 |

Data are the mean ± SD for n = 8 mice in each group.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

What is claimed is:

1. A method for increasing muscle function in a subject, comprising:
providing the subject with a composition comprising an effective amount of paraxanthine.

2. The method of claim 1, wherein paraxanthine is present in the composition in amount from about 25 mg to about 600 mg.

3. The method of claim 2, wherein paraxanthine is present in the composition in amount from about 50 mg to about 400 mg.

4. The method of any of claim 1, wherein the composition further comprises one or more compounds selected from the list consisting of: isoleucine, leucine, and valine, histidine, isoleucine, leucine, lysine, methionine, phenylalanine, threonine, tryptophan, valine, creatine, arginine, cysteine, glutamine, glycine, proline, carnitine, creatinol, beta-alanine, ATP, protein, peptides, and beta-hydroxy beta-methylbutyrate.

5. The method of claim 1, wherein the composition is substantially free of caffeine.

6. The method claim 1, wherein the composition is substantially free of caffeine, taurine, tyrosine, and 1-methylxantine.

7. The method of claim 1, wherein the composition is administered in conjunction with a strength training regimen.

8. The method of claim 7, wherein the muscle function is increased by from about 10% to about 20%, relative to a subject receiving a control composition without paraxanthine.

9. A method of increasing muscle strength and/or muscle size comprising administering to a subject in need thereof with a composition comprising an effective amount of paraxanthine.

10. The method of claim 9, wherein paraxanthine is present in the composition in amount from about 20 mg to about 600 mg.

11. The method of claim 10, wherein paraxanthine is present in the composition in amount from about 50 mg to about 400 mg.

12. The method of claim 9, wherein the composition is substantially free of caffeine.

13. The method claim 9, wherein the composition is substantially free of caffeine, taurine, tyrosine, and 1-methylxantine.

14. The method of claim 9, wherein the composition is administered in conjunction with a strength training regimen.

15. The method of claim 14, wherein muscle strength is increased from about 10-25% relative to a subject receiving a control composition without paraxanthine.

16. The method of claim 14, wherein muscle size is increased from about 10-20% relative to a subject receiving a control composition without paraxanthine.

17. The method of claim 14, wherein nitric oxide (NO) signaling is increased from about 90-100% relative to a subject receiving a control composition without paraxanthine.

18. The method of claim 14, wherein muscle glycogen levels are increased from about 10-30% relative to a subject receiving a control composition without paraxanthine.

19. A method of treating or preventing muscle atrophy in a subject in need thereof of comprising administering to the subject and effective amount of paraxanthine.

20. The method of claim 19, wherein the subject has been diagnosed with, or is at risk of developing, sarcopenia or cachexia.
